(19) United States
(12) Patent Application Publication
(10) Pub. No.: US 2003/0113270 A1
(43) Pub. Date: Jun. 19, 2003

(54) Vasoactive intestinal peptides for glaucomatous retinopathy

(76) Inventor: Abbot F. Clark, Arlington, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/313,571

(22) Filed: Dec. 6, 2002

Related U.S. Application Data
(60) Provisional application No. 60/340,152, filed on Dec. 14, 2001.

(51) Int. Cl. A61K 38/08; A61K 38/06; A61L 9/04

(52) U.S. Cl. 424/45; 514/16; 514/17; 514/18

(57) ABSTRACT

The present invention provides methods for treatment of glaucoma and other retinal diseases comprising the use of compositions containing vasoactive intestinal peptides.

This application claims priority from U.S. Provisional Application Serial No. 60/340,152 filed Dec. 14, 2001.

### BACKGROUND OF THE INVENTION

1. Field of the Invention

The present invention is directed to the use of vasoactive intestinal polypeptides (VIP), VIP peptides and VIP mimetics for the treatment of glaucoma and other retinal diseases.

2. Description of the Related Art

“Glaucomas” are a group of debilitating eye diseases that are the leading cause of preventable blindness in the United States and other developed nations. Primary Open Angle Glaucoma (“POAG”) is the most common form of glaucoma. The disease is characterized by the degeneration of the trabecular meshwork, leading to obstruction of the normal ability of aqueous humor to leave the eye without closure of the space (e.g., the “angle”) between the iris and cornea (Vaughan, D. et al., (1992)). A characteristic of such obstruction in this disease is an increased intraocular pressure (“IOP”), resulting in progressive visual loss and blindness if not treated appropriately and in a timely fashion. The disease is estimated to affect between 0.4% and 3.3% of all adults over 40 years old (Leske, M. C. et al. (1986); Bengtsson, B. (1989); Strong, N. P. (1992)). Moreover, the prevalence of the disease rises with age to over 6% of those 75 years or older (Strong, N. P., (1992)).

Glaucoma affects three separate tissues in the eye. The elevated IOP associated with POAG is due to morphological and biochemical changes in the trabecular meshwork (TM), a tissue located at the angle between the cornea and iris. Most of the nutritive aqueous humor exits the anterior segment of the eye through the TM. The progressive loss of TM cells and the build-up of extracellular debris in the TM of glaucomatous eyes leads to increased resistance to aqueous outflow, thereby raising IOP. Elevated IOP, as well as other factors such as ischemia, cause degenerative changes in the optic nerve head (ONH) leading to progressive “cupping” of the ONH and loss of retinal ganglion cells and axons. The detailed molecular mechanisms responsible for glaucomatous damage to the TM, ONH, and the retinal ganglion cells are unknown.

Current glaucoma therapy is directed to lowering IOP, a major risk factor for the development and progression of glaucoma. These therapies lower IOP, but they do not directly address the pathogenic mechanisms, and the disease continues to progress. At least half of patients with glaucoma are undiagnosed, and by the time patients are diagnosed with glaucoma, they have already lost approximately 40% of their retinal ganglion cells. Almost all current glaucoma therapy is directed to lowering elevated IOP, a major risk factor for the development of glaucomatous optic neuropathy. However, the actual loss of vision in glaucoma is due to the death of retinal ganglion cells, and to date, there is no effective treatment directed towards protecting these cells. In view of the importance of glaucoma, and the at least partial inadequacies of prior methods of treatment, it would be desirable to have an improved method of treating glaucoma which would address the underlying causes of its progression.

### SUMMARY OF THE INVENTION

The present invention overcomes these and other drawbacks of the prior art by providing an improved method for the protection of retinal ganglion cells in glaucomatous optic neuropathy. The method of the invention comprises administering to a patient in need thereof a pharmaceutically effective amount of a composition containing at least one vasoactive intestinal peptide or an analog thereof. In preferred embodiments, the vasoactive intestinal peptide for use in the method of the invention is St-KKYL-NH₂, SNV, St-KKYV-NH₂, St-KKYdA-NH₂, St-KKYNle-NH₂, St-KVYL-NH₂, St-VKKYL-NH₂, St-KKYLN-NH₂, St-KKYLNS-NH₂, St-KKYLNS₁-NH₂, St-KKYLNSIL-NH₂ or St-NSILN-NH₂.

In preferred embodiments, the vasoactive intestinal peptides are present in the compositions for use in the methods of the invention in a range of from 0.1%-2.0% for use in a topical ocular formulation, from 1.0%-5.0% for sub-tenon's injection, from 1.0% -10% for use in an intraocular insert device or from 0.5%-5.0% for intravitreal injection.

### DETAILED DESCRIPTION PREFERRED EMBODIMENTS

The trabecular meshwork has been proposed to play an important role in the normal flow of the aqueous humor, and has been presumed to be the major site of outflow resistance in glaucomatous eyes. Human trabecular meshwork (HTM) cells are endothelial like cells which line the outflow channels by which aqueous humor exits the eye. Altered synthetic function of the cells may be involved in the pathogenesis of steroid glaucoma and other types of glaucoma.

Almost all current glaucoma therapy is directed to lowering elevated intraocular pressure (IOP), a major risk factor for the development of glaucomatous optic neuropathy. However, the actual loss of vision in glaucoma is due to the death of retinal ganglion cells. To date, there is no effective treatment directed toward protecting these cells.

The present invention stems in part from the recognition that vasoactive intestinal polypeptide (VIP) and peptide analogs of VIP are neuroprotective in in vitro and in vivo models of β-amyloid toxicity to neuron and glial cells. VIP-related peptide analogs protected rat cerebral cortical cultures (mixed cultures containing both neuronal and glial cells) from cytotoxicity induced by the neurotoxin β-amyloid peptide. In addition, prophylactic administration of VIP-peptide analogs restored brain choline acetyltransferase activity in Apo E-deficient mice and ameliorated learning and memory impairment in Apo E-deficient as well as cholinergically impaired animals (Gozes et al., 1999).

Gozes et al. report that the 28 amino acid neuropeptide, VIP, has been shown to provide neuroprotection and that a lipophilic VIP derivative exhibited potencies 100-fold higher than VIP. Stearyl-norleucine¹⁷-VIP (SNV) has been shown to be 100-fold more potent than VIP in promoting neuronal survival. Gozes tested other stearyl-VIP-related peptide analogs and found them to be neuroprotective as well. Gozes et al.'s studies were performed in Alzheimer's disease-relevant models. There is no suggestion that such peptides would be useful in the treatment of glaucoma.

The present inventor has discovered that VIP-related peptides containing an N-terminal stearic acid attachment and an amidated C terminus may provide protection to retinal ganglion cells in patients suffering from glaucoma. In particular, it is contemplated that peptides having the following structures will be useful in the methods of the invention: St-KKYL-NH₂, SNV, St-KKYV-NH₂, St-KKYdA-NH₂, St-KKYNle-NH2, St-KYL-NH₂, St-VKKYL-NH₂, St-KKYLN-NH₂, St-KKYLNS-NH₂, St-KKYLNSI-NH₂, St-KKYLNSIL-NH₂ and St-NSILN-NH₂.

The agent may be delivered directly to the eye (for example: topical ocular drops or ointments; slow release devices in the cul-de-sac or implanted adjacent to the sclera or within the eye; periocular, conjunctival, sub-Tenons, intracameral or intravitreal injections) or parenterally (for example: orally; intraveneous, subcutaneous or intramuscular injections; dermal delivery; etc.) using techniques well known by those skilled in the art. The following are examples of possible formulations embodied by this invention.

| | wt. % |
|---|---|
| (a) Topical ocular formulation | |
| VIP | 0.1-2.0 |
| HPMC | 0.5 |
| Sodium chloride | 0.8 |
| BAC | 0.01% |
| EDTA | 0.01 |
| NaOH/HCl | qs pH 7.4 |
| Purified water | qs 100 mL |
| (b) Topical ocular formulation | |
| VIP | 1.0-5.0 |
| HPMC | 0.5 |
| Sodium chloride | 0.8 |
| BAC | 0.01 |
| EDTA | 0.01 |
| NaOH/HCl | qs pH 7.4 |
| Purified water | qs 100 mL |
| (c) Intravitreal injection | |
| VIP | 0.5-5.0 |
| HPMC | 0.5 |
| Sodium chloride | 0.8 |
| NaOH/HCl | qs pH 7.2 |
| Purified water | qs 100 mL |

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

VIP-related peptides analogs are tested for neuroprotective activity in rat retinal ganglion cell (RGC) cultures as previously described (Pang et al. (1999)). The fluorescent dye, Di-I, is injected into the superior colliculi of neonatal rats 2-4 days prior to removal, dissociation, and culture of the retinal cells. The cultured RGCs are exposed to various cytotoxic insults (such as excessive glutamate, oxygen-glucosa deprivation, or withdrawal of serum from the media) in the absence or presence of VIP peptides, and RGC survival is assessed by counting the selectively labelled Di-I fluorescent cells. Similar in vitro assays can be conducted using surrogate cell lines such as dSHSY5Y cells.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Bengtsson, B. (1989).

Gozes et al., “Mapping the active site in vasoactive intestinal peptide to a core of four amino acids: Neuroprotective drug design,” PNAS 96:4143-4148 (1999).

Leske, M. C. et al. (1986)

Pang et al., INVEST. OPHTHALMOL. VIS. Sci., 40(6):1170-1176 (1999).

Strong, N. P. (1992).

Vaughan, D. et al., (1992).

We claim:

1. A method for the protection of retinal ganglion cells in glaucomatous optic neuropathy, said method comprising administering to a patient in need thereof a pharmaceutically effective amount of a composition comprising a vasoactive intestinal peptide or an analog thereof.

2. The method of claim 1, wherein the vasoactive intestinal peptide may be selected from the group consisting of St-KKYL-NH₂, SNV, St-KKYV-NH₂, St-KKYdA-NH₂, St-KKYNle-NH₂, St-KVYL-NH₂, St-VKKYL-NH₂, St-KKYLN-NH₂, St-KKYLNS-NH₂, St-KKYLNSI-NH₂, St-KKYLNSIL-NH₂, and St-NSILN-NH₂.

3. The method of claim 1, wherein the administering is by intraocular injection, implantation of a slow release delivery device, topical administration, or oral administration.

4. The method of claim 2, wherein the administration is by intranasal administration.
